(19) United States
(12) Patent Application Publication
(10) Pub. No.: US 2007/0179432 A1
(43) Pub. Date: Aug. 2, 2007

(54) Endoscopic Apparatus Provided With Pressure Relief Arrangement

(75) Inventors: Yakov BAR OR, Haifa (IL); Dan OZ, Even Yehuda (IL); Victor Levin, Haifa (IL); Menachem SHAVIT, Misgav (IL)

Correspondence Address: BRUCE E. LILLING;LILLING & LILLING PLLC, P.O. BOX 560, GOLDEN BRIDGE, NY, 10526, US

(73) Assignee: STRYKER GI LTD., Haifa (IL)

(21) Appl. No.: 11/616,066

(22) Filed: Dec. 26, 2006

Related U.S. Application Data
(60) Provisional application No. 60/597,928, filed on Dec. 27, 2005.

(51) Int. Cl. A61M 1/00

(52) U.S. Cl. 604/30

(57) ABSTRACT

An endoscopic apparatus for endoscopic examination of a body channel or cavity is described. The endoscopic apparatus comprises an insertion tube with at least one channel, which is in flow communication with the body channel or cavity. The endoscopic apparatus comprises also a pressure relief arrangement fitted with a valve, which is in flow communication with the body channel or cavity. The valve is suitable for preventing an inner pressure within the body channel or cavity to exceed a threshold value.

### BACKGROUND OF THE INVENTION

1. Field of the Invention

The present invention relates generally to the field of endoscopy and specifically to endoscopic apparatus used for colonoscopic procedures during which a flexible tube is inserted through the rectum into the colon for examination of the colon's interior for abnormalities and the colon is insufflated. More particularly, the present invention refers to a pressure relief means, which prevents the pressure within the colon from exceeding a certain level. By virtue of this relief means, it is possible to prevent barotrauma injuries, like rupturing of the colon, perforation of the cecum, etc

2. Summary of the Prior Art

The consequences of barotrauma caused during colonoscopic procedure are well documented; see for example an article “A retrospective analysis of cecal barotrauma caused by colonoscope air flow and pressure”, Gastrointestinal Endoscopy, 2005, volume 61, No. 1, 37-45.

In the industry, there are various known endoscopes for medical or industrial applications, which are provided with means for measuring inner pressure during the endoscopic procedure, but not for releasing the pressure. Machida (U.S. Pat. No. 4,411,257) discloses that the inner pressure can be seen in the eye lens zone of the inspection endoscope; Slanetz (U.S. Pat. No. 4,469,091) discloses that the sheath and electrodes are attached to the ohmmeter so that, when the instrument contacts the wall of the colon, the pressure is measured, so as to avoid areas with too high a pressure; Sugrue (U.S. Pat. No. 5,433,216) discloses a tonometric catheter having one or more pressure transmitting chambers for sensing the internal pressure, such as the internal abdominal pressure; and, Kulik (U.S. Pat. No. 4,893,634) discloses an endoscope with an open-end capillary tube with a pressure measuring instrument on one of its ends.

It is also known to use endoscopic apparatuses, in which the insertion tube is covered by an inflatable disposable sleeve, such as disclosed by Eizenfeld (WO 2004/016299; International patent application PCT/IL2003/000661) and Bar-Or (WO 2005/110204; International patent application PCT/IL2005/000425). While each relies on the use of some type of gas to inflate the disposable sleeve, neither provides any means for releasing gas and thereby preventing barotrauma.

Since neither the conventional endoscopes without an inflatable disposable sleeve nor the endoscopic apparatuses with such inflateable disposable sleeves are provided with a means for pressure relief, Barotrauma can occur and an inadvertent injury may take place during the endoscopic procedure when pressure of air is supplied to the hollow body organ.

There is a known inflatable sheath device for an endoscope as disclosed in Takahashi (U.S. Pat. No. 5,105,800). This device comprises a sheath, which is inflatable with a gas that is pumped into the inside of the sheath to allow an insert tube of the endoscope to be removed from the sleeve. The endoscope is provided also with a gas pressure control device, which has a bleeder communicating with the gas supply passage and open upwardly to the atmosphere. A ball valve is placed on the bleeder from the upper side thereof to close the bleeder when the pressure of air in the sleeve is less than the load applied on the ball valve. When this pressure exceeds a certain limit, corresponding to the applied load, the ball valve should float up and open the bleeder, thereby releasing the air to the outside and preventing the sleeve from bursting. Unfortunately this sheath device is intended merely for easy removal of the insert tube from the sleeve after the tube has been withdrawn out of a hollow organ of the patient's body. The easy removal is achieved due to inflation of the sheath. The gas pressure control device employed in this sheath device is designed to prevent bursting of the sheath during inflation and is not intended for and is not capable of preventing barotrauma during the endoscopic procedure when the insertion tube is located inside the hollow organ and the pressure of air is supplied into the hollow organ.

### SUMMARY OF THE INVENTION

The object of the present invention is to provide an endoscopic apparatus with a relief arrangement capable of preventing injuries caused during the endoscopic procedure when the pressure of air is supplied through the insertion tube to the hollow organ or to the body channel. In the following description, the body channel means any body passage, cavity or organ, whose interior is to be examined during the endoscopic procedure.

One of the objects of the invention is to provide a new pressure relief arrangement for an endoscopic apparatus, which is convenient and simple both in operation and in maintenance.

A further object of the invention is to provide a pressure relief arrangement for an endoscopic apparatus, which has a means for measuring pressure in the body cavity during the endoscopic procedure.

Still a further object of the invention is to provide a pressure relief arrangement for an endoscopic apparatus, which is capable of automatically relieving pressure from the body channel when this pressure exceeds a certain preset level or to relieve pressure upon receiving a control signal from a system control unit.

For a better understanding of the present invention as well of its benefits and advantages, reference will now be made to the following description of its embodiments taken in combination with the accompanying drawings.

### BRIEF DESCRIPTION OF THE SEVERAL VIEWS OF THE DRAWINGS

FIG. 1 is a general schematic view of the prior art endoscopic apparatus provided with a disposable inflatable sleeve.

FIG. 2 is a diagrammatical view of the control system employed in the endoscopic apparatus shown in FIG. 1.

FIG. 3a shows a first embodiment of the pressure relief arrangement, which can be employed in the endoscopic apparatus presented in FIG. 1, wherein the fluid control system is provided with a check valve retrofitted in the suction line between a pinch valve and the connector.

FIG. 3b shows a second embodiment of the pressure relief arrangement, which can be employed in the endoscopic apparatus presented in FIG. 1, wherein the fluid control system is provided with a means for measuring pressure in the body channel and with an electrically controlled relief valve.

FIG. 3c shows a third embodiment of the pressure relief arrangement, which can be employed in the endoscopic apparatus presented in FIG. 1, wherein the means for measuring pressure and the relief valve are retrofitted in the insufflation.

FIG. 4a shows an embodiment of the pressure relief arrangement, which can be employed in conventional endoscopic apparatus, which is not provided with an inflatable disposable.

FIG. 4b shows an embodiment of the pressure relief arrangement, which can be employed in conventional endoscopic apparatus, wherein the operating handle is provided with a means for measuring the pressure and with an electrically controlled relief valve.

### DETAILED DESCRIPTION OF THE PREFERRED EMBODIMENT

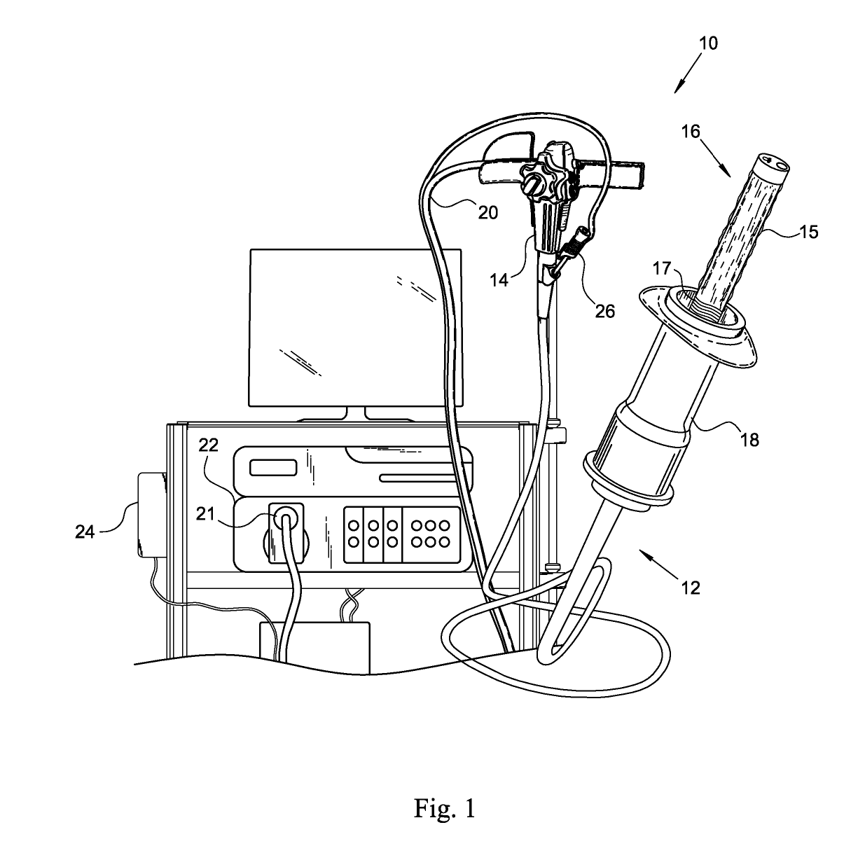

With reference to FIG. 1 a prior art endoscopic apparatus, preferably a colonoscopic apparatus 10, is shown with its following main components. The apparatus comprises an endoscope having an insertion tube with its proximal section 12 connected to an operation handle 14 and with its distal section 16 inserted in and protruding from a disposable dispenser 18. An example of such an apparatus and a general explanation of its construction and functioning can be found in Eizenfeld (WO 2004/016299; International patent application PCT/IL2003/000661) and Bar-Or (WO 2005/110204; International patent application PCT/IL2005/000425), whose disclosures are hereby incorporated herein by reference

It is shown also in FIG. 1 that a disposable inflatable sleeve covers the distal region of the endoscope. That part of the sleeve, which is seen in FIG. 1, comprises a frontal noninflatable portion 15 and a rear folded portion 17. The frontal noninflatable portion 15 covers the distal section 16 of the endoscope and its head. During the procedure when the endoscope advances within the colon, the frontal portion 15 does not inflate, whereas the rear portion 17 covers the insertion tube and unfolds when air, or other fluid medium, is pumped into and inflates the sleeve. The endoscope is propelled within the body passage when the sleeve is being inflated.

It should be appreciated, however, that the present invention is not limited merely to colonoscopy as such and merely to the endoscopes, which are provided with inflatable sleeve. It can be employed in any other endoscopic apparatus used for a medical procedures requiring insertion of a probe in a body passage for inspection of its interior and in which the body passage is inflated.

It is seen also in FIG. 1, that the handle is connected by an umbilical cord 20 to a multifunctional connector 21, which is plugged into a system control unit (SCU) 22.

Within the SCU there is provided a source of compressed air for inflating the sleeve and for insufflation the body passage.

Proximate to the SCU, an irrigation flask 24 is provided, which is filled with water or other liquid, to be supplied via insertion tube into the colon for irrigation or for other purpose.

It is not shown specifically, but should be appreciated that appropriate channels extend along the umbilical cord. Among these channels are a channel for inflating the sleeve, an insufflation channel for insufflation the body passage, an irrigation channel for supplying water to the body passage and cleaning the optical head and a vacuum channel for suction from the body channel and also for insertion of surgical tools such as biopsy forceps.

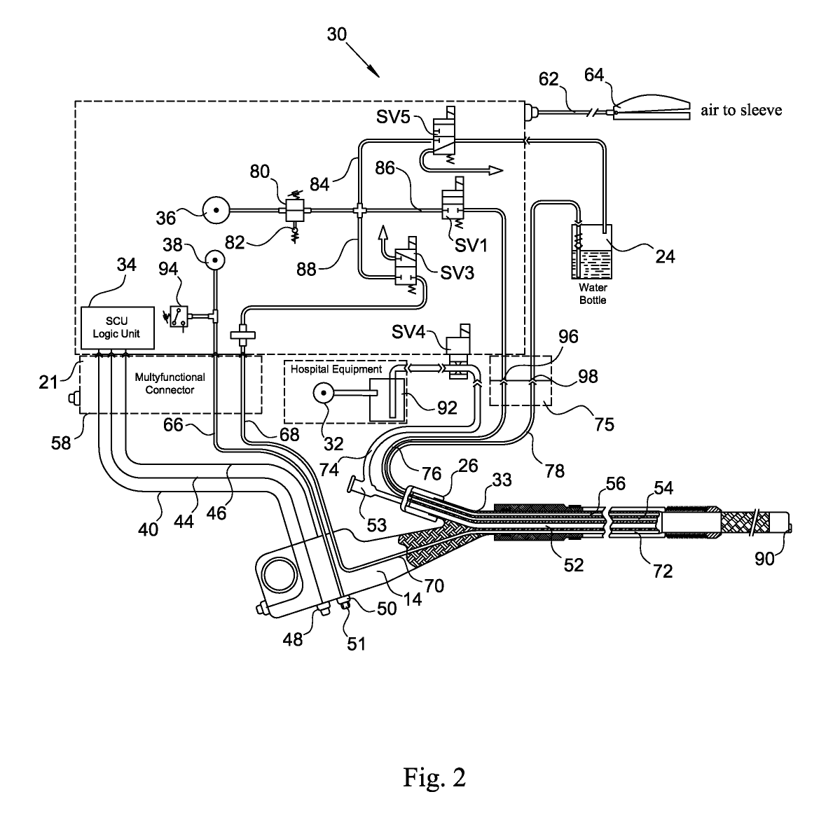

The SCU is one of the main parts of the control system and it will be explained in more details further in connection with FIG. 2.

One should also bear in mind that within the insertion tube are also provided various devices, which are necessary for proper functioning of the colonoscopic apparatus. These devices are known per se. Among such devices one can mention e.g. vertebrae and strings, which can be manipulated by the operation handle.

It is not seen in FIG. 1, but should be appreciated, that along the insertion tube extends a multilumen tubing with appropriate passages for supplying water, as required for irrigation of the colon, air as required for insufflation and vacuum as required for suction.

The multilumen tubing also allows introduction of surgical instruments into the colon as might be required during the colonoscopic procedure. The multilumen tubing extends through the entire length of the insertion tube, passes the handle and is connected to a dedicated connector 26, which is detachably connectable to a lateral port provided on the handle, so as to connect the proximal end of the multilumen tubing with channels extending along the umbilical cord.

In FIG. 2 a diagram explaining fluid control system of the endoscopic apparatus is seen. This fluid control system is intended preferably for use with the endoscopic apparatus provided with a disposable sleeve. The fluid control system is designated by reference numeral 30 and its main component, i.e. the SCU is defined schematically by a dotted line. The SCU controls supply of air, water and vacuum as required for proper functioning of the colonoscopic apparatus 10.

Some external components of the fluid control system, namely irrigation flask 24 and vacuum pump unit 32, are also seen. As a suitable source of vacuum one could use available hospital equipment capable of producing the necessary suction from the body passage through the multilumen tubing.

The multilumen tubing is also schematically depicted in FIG. 2 and it is designated by reference numeral 33. Within the SCU are provided the necessary electronic, pneumatic and hydraulic components, e.g. a logic unit 34, a first pump 36 and a second pump 38 for supplying compressed air.

Various valves are also shown as will be explained later on. It is not shown specifically but should be appreciated that a dedicated power supply means can be also provided within the SCU as required for activation the valves and energizing the logic unit.

In practice the first pump 36 should be capable to supply air under pressure 0.5-0.7 bar with a flow rate 3-5 liters per minute. This pump is intended to supply compressed air for insufflation the body channel, for inflating the sleeve and for supplying water from the irrigation flask. The second pump should be capable of supplying air under pressure 0.3 bar with a flow rate 2 liter per minute. This pump is intended for supplying air to the operating handle. The operating handle has an opening for releasing the air. The purpose of this arrangement will be explained further.

The logic unit is also electrically connected by signal lines 40, 44, 46 to respective components of the operating handle. In particular lines 44, 46 lead to electrical control buttons 48 and 50, which are provided on the handle. Control button 48 enables controlling of suction through a channel 52 made in the multilumen tubing. This channel functions either as a suction channel (when vacuum is supplied therethrough) or as a working channel when it is required to insert a surgical tool in the body channel through a port 53.

Control button 50 enables controlling of air supply to the body passage through a dedicated insufflation channel 54 provided in the multilumen tubing. This button also enables the supply of water to the body passage through a dedicated irrigation channel 56 provided in the multilumen tubing.

A through going opening 51 is provided in the button 50. This opening can be closed or opened by the doctor's finger during operating of the handle. The through going opening is in flow communication with the second pump 38.

The multifunctional connector 21 allows electrical connection of the SCU with the signal lines 44 and 46. The logic unit 34 is electrically connected by a line 62 with a foot pedal 64, which upon pressing generates a signal, which triggers inflation of the sleeve.

In FIG. 2 are also seen tubes 66, 68, which provide flow communication between the SCU and the operating handle. The tubes are detachably connected to the SCU by virtue of the same multifunctional connector 21.

It is seen that tube 66 serves for supplying pressurized air from pump 38 to opening 51 in the button 50. It is also seen that tube 68 supplies pressurized air from pump 36 to the handle. Within the handle there is provided a passage 70 through which compressed air from pump 36 proceeds to a channel 72 extending through the insertion tube. This channel is intended for supplying air required for inflating the sleeve.

It is shown also in FIG. 2 that the multilumen tubing is also in flow communication with the SCU via channels 74, 76, 78. These channels are connected to the connector 26 provided at the side extension of the handle. The connector is also fitted with the port 53. The channels 74, 76, 78 respectively supply vacuum to the working channel 52, air to the insufflation channel 54 and water to the irrigation channel 56.

A common connector 75 is provided for simultaneous bringing channel 76 in fluid communication with pump 36 and channel 78 with irrigation flask 24. In accordance with one of the aspects of the present invention the common connector and channels 74, 76, 78 are disposable items. Furthermore, the channels 76 and 78 are immediately connectable and disconnectable to the respective source of air and water without the necessity to connect/disconnect the tubes one by one as it could be required if separate connectors would be used each line. This provision renders the setting up of the whole control system very simple, convenient and fast.

It is not shown specifically but should be appreciated that connector 26 could be arranged at the flask, e.g. in its cover.

Within the SCU are mounted hydraulic and pneumatic components of the system. These components are necessary for controlled supply of the fluid medium to the colonoscope.

The fluid medium is supplied by the following supply lines: line A for supplying compressed air from the first pump 36 to the sleeve, to the multilumen tubing and to the flask; line B for supplying vacuum produced by vacuum pump unit 32 to the multilumen tubing; line C for supplying of compressed air from pump 38 to the handle; and line D for supplying of water from flask 24 to the multilumen tubing.

It is seen, for example, that line A comprises a pressure regulator 80 with a safety valve 82 for keeping the pressure supplied by pump 36 within a narrow range of 0.5-0.7 bar. The pressurized air proceeds via ducts 84, 86 to respective normally shut off solenoid valves SV5, SV1. These valves, when opened, enable supply of pressurized air from the pump either to flask 24, or to channel 76.

As soon as pressurized air is supplied to the flask, water within the flask is urged to proceed via supply tube 78 to the irrigation channel. From here water is ejected through an aperture in the distal end of the insertion tube and is directed outside by a sprinkler means 90 provided at the distal end of the insertion tube. In practice water is ejected from the flask with a flow rate of at least 1 cc per second. It can be readily appreciated that pressure in the flask is not maintained permanently, but only when it is required to supply water for irrigation.

The line B comprises a suction bottle 92 and a suction valve SV4, which is conventional pinch valve capable to selectively release the tube 74 passing therethrough. Upon pressing suction button 48 on the operating handle this pinch valve can be actuated.

It should be appreciated that all valves are electrically connected to the logic unit and are controlled thereby.

The line C comprises a pressure sensor 94, which senses air pressure in the line 66. The pressure sensor is electrically connected to the logic unit.

As soon as doctor closes with his/her finger the through going opening 51, the air pressure in line 66 increases above a certain preset level and the sensor generates and sends to the logic unit a signal.

Upon receiving this signal the logic unit opens valve SV1 and pressurized air is supplied via line 76 to the insufflation channel of the multilumen tubing.

Check valves 96, 98 can be provided in the common connector 75. In practice these check valves can be conventional ball valves. The check valves are installed in the respective lines A and B. The check valves are intended for preventing back flow of air and water into the SCU through respective channels 76 and 78.

The explained above fluid control system is preferably employed in the endoscopic apparatuses, which are provided with disposable inflatable sleeve.

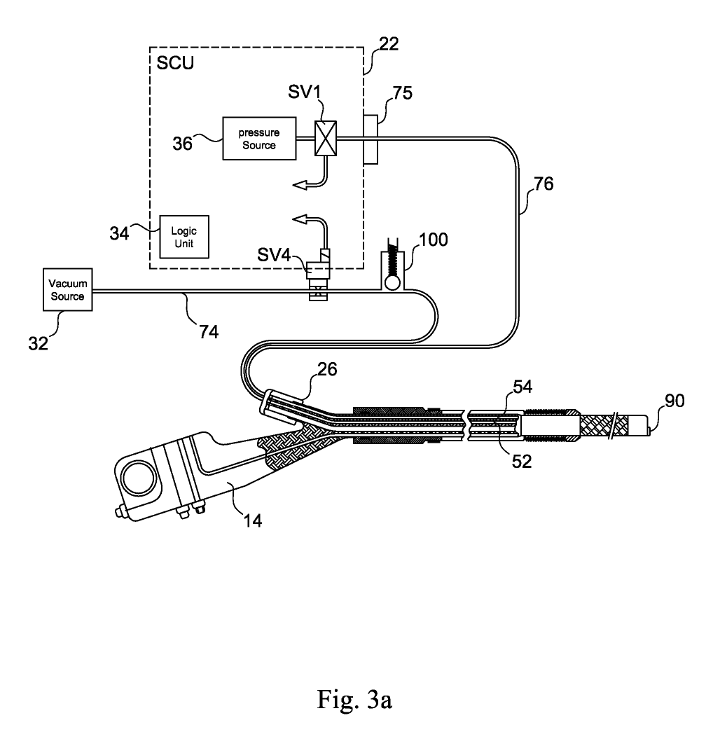

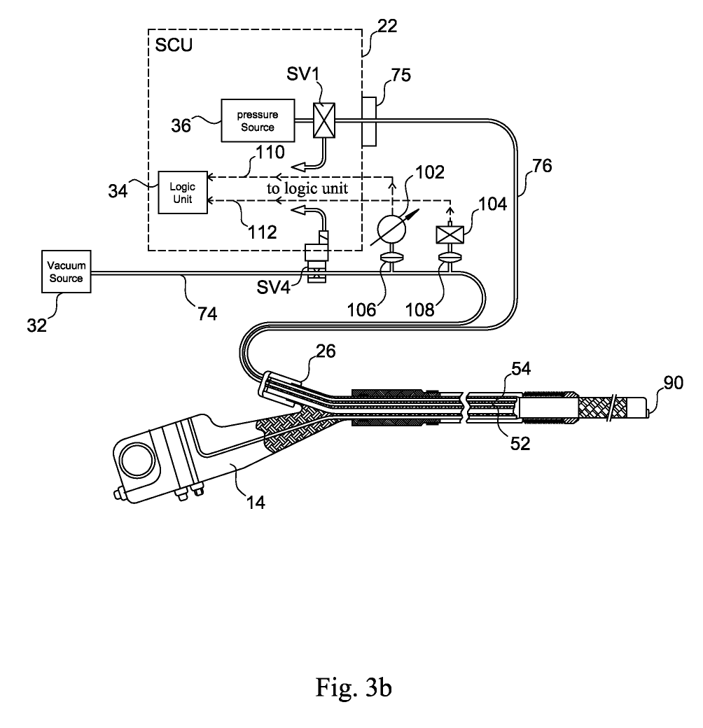

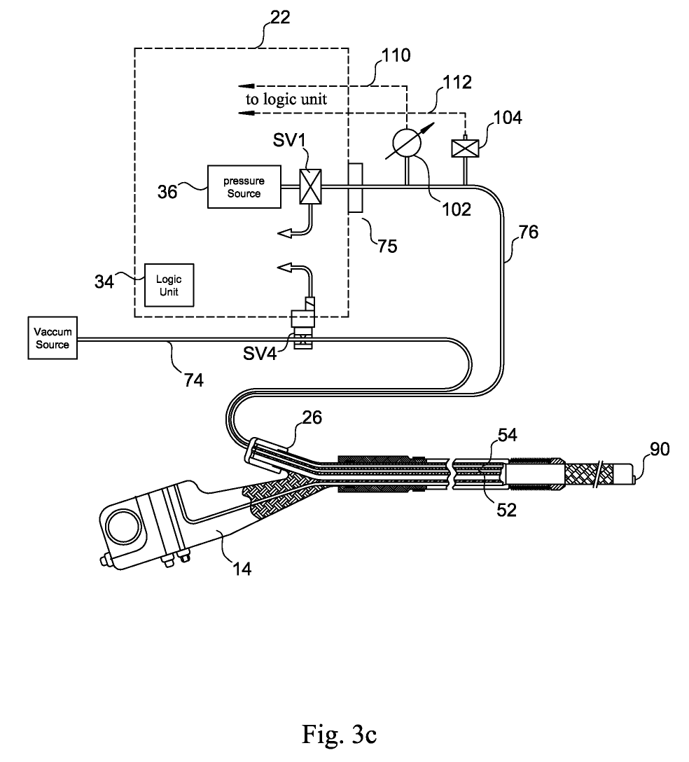

In accordance with the present invention this fluid control system is provided with a pressure relief arrangement as will be disclosed further with reference to FIGS. 3a, 3b and 3c. For the sake of brevity only a fragment of the endoscopic apparatus is shown. This fragment schematically depicts operating handle, insertion tube, system control unit, vacuum source, suction line and insufflation line.

Referring to FIG. 3a a first embodiment of the pressure relief arrangement will be explained. According to this embodiment the fluid control system of the colonoscopic apparatus shown in FIG. 1 is provided with a check valve 100 retrofitted in the suction line 74 between pinch valve SV4 and connector 26.

One port of the check valve is in fluid communication with the suction line and the opposite port is open to atmosphere. In practice this check valve can be a ball valve, which is set to automatically open as soon as pressure in the suction line 74 exceeds some threshold.

When the colonoscopic apparatus is in the suction mode the pinch valve SV4 is open and vacuum in the channel 74 keeps the check valve closed.

When there is no need in suction, the pinch valve is closed and suction channel 74 is in fluid communication with the body channel through working channel 52 extending along the multilumen tubing and through connector 26 at the operating handle 14. In this situation the check valve senses inner pressure in the body channel. The check valve is preset to a certain threshold and will automatically release the inner pressure in the body channel to atmosphere as soon as the inner pressure exceeds the set threshold. In practice the threshold is set to 0.2 bar.

Referring now to FIG. 3b a second embodiment of the pressure relief arrangement will be explained. In accordance with this embodiment the fluid control system of the endoscopic apparatus shown in FIG. 1 is provided with a means 102 for measuring pressure in the body channel and with separate electrically controlled relief valve 104.

It is advantageous if the means 102 for measuring pressure and the valve 104 is fitted with appropriate biologic filter 106,108 for preventing contamination which may origin from the channel 74.

In FIG. 3b the means 102 and the relief valve 104 are located in the suction line between the pinch valve SV4 and connector 26.

Once the pinch valve is closed the means 102 senses and measures inner pressure in the body channel through suction channel 74 and connector 26. This pressure is built during the endoscopic procedure when there is no suction and the body channel is insufflated or when the sleeve is inflated. For measuring the inner pressure one can use any suitable means, e.g. a pressure transducer or a manometer, capable of providing an output reading that corresponds to the inner pressure in the body channel. A solenoid valve can be used as suitable electrically controllable relief valve 104.

A first control line 110 is provided, which electrically connects the means 102 for measuring pressure with the logic unit 34. A second control line 112 is provided, which electrically connects the valve 104 with the logic unit 34. The output reading from the means 102 proceeds to logic unit 34 where it is permanently monitored and compared with a stored threshold value.

If the instant value exceeds the threshold value, the logic unit generates a control signal for opening the valve. If required the monitored value can be displayed on a monitor. Alternatively or additionally the control signal can be sent from the logic unit to the valve SV1, which upon receiving the signal closes line 76 and terminates insufflation provided by pump 36.

In FIG. 3c is shown still further embodiment of the present invention. In accordance with this embodiment the means 102 for measuring pressure and relief valve 104 are located in the insufflation line 76 between the common connector 75 and connector 26. In this embodiment there is no need in biological filters, since flow of air is always maintained from the SCU to the body channel. Similarly to the previous embodiments the means 102 for measuring pressure is connected to the logic unit by first control line 110 and relief valve 104 is connected to the logic unit by second control line 112. The control signal generated by the logic unit is sent to relief valve 104, which will release the pressure in line 76 to atmosphere. Alternatively or additionally the control signal can be sent to valve SV1, which upon receiving the signal closes line 76 and terminates insufflation.

It should be borne in mind that it is not compulsory that the means 102 for measuring pressure and relief valve 104 are located in the same line. One can contemplate a situation, in which one of these two components is retrofitted in the suction line, while the other component is retrofitted in the insufflation line. It is essential however, that both components would be in flow communication with the body channel either through suction channel 52 or through insufflation channel 54.

Up to now the present invention has been disclosed in connection with the endoscopic apparatus provided with inflatable disposable sleeve and with electrically controlled buttons. The present invention, however, is not restricted merely to such an apparatus.

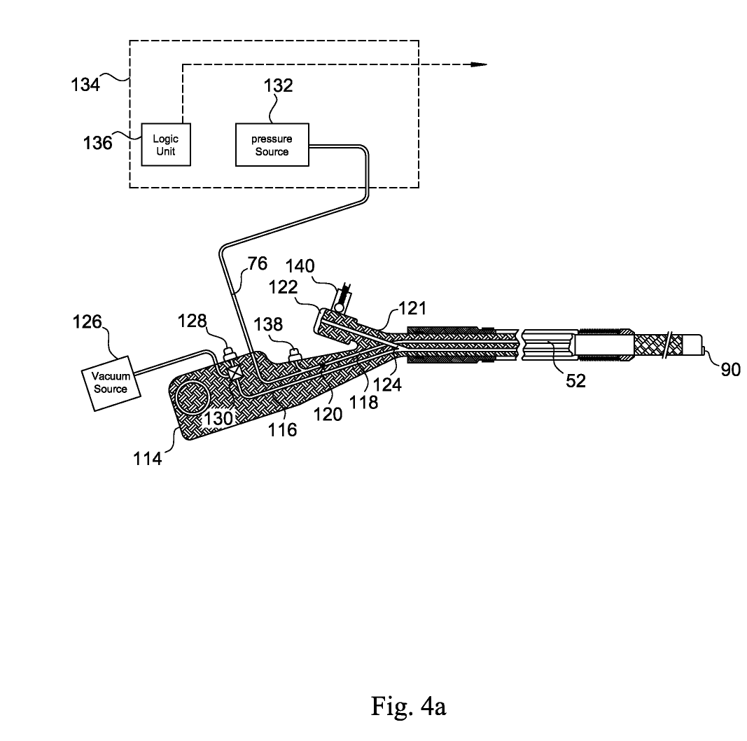

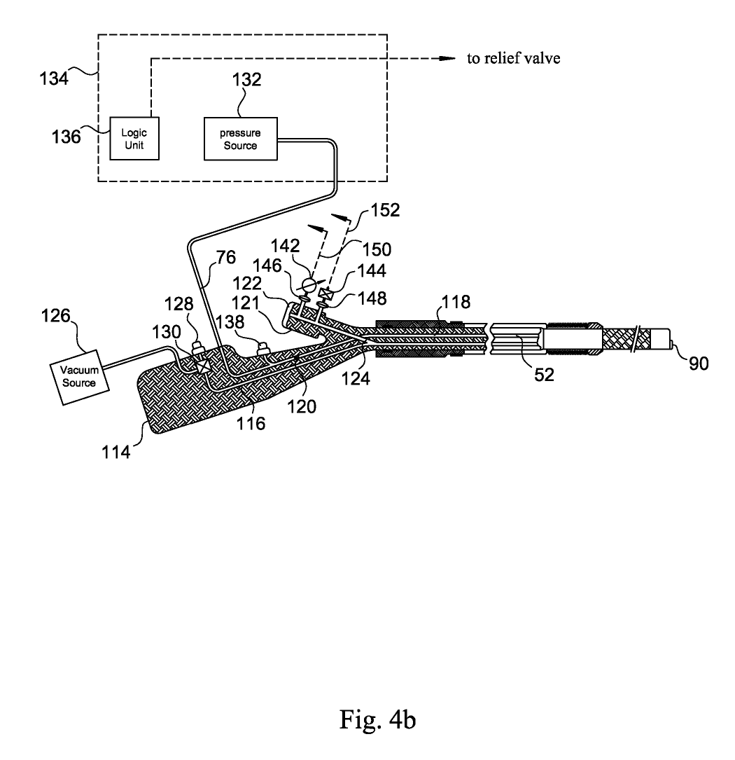

Attention is called now to still further embodiments shown in FIG. 4a and FIG. 4b. These embodiments refer to a conventional endoscopic apparatus, which is not provided with inflatable disposable sleeve. In this embodiment mechanically controlled buttons trigger insufflation and suction.

For the sake of brevity merely a fragment of such conventional endoscopic apparatus is shown. The fragment depicts operating handle, insertion tube, system control unit, vacuum source, suction line and insufflation line.

It is seen an operating handle 114 with extending therealong a suction channel 116 and an insufflation channel 118. A check valve 120 is provided within the insufflation channel.

A lateral port 121 is provided at the operating handle for entering a surgical tool thereinto and advancing the tool further through the suction channel. A seal 122 seals the lateral port. A working channel 124 extends along the lateral port and communicates with the suction channel 116.

The suction channel is provided with an inlet port, which is in flow communication with a vacuum source 126. A suction button 128 is provided at the operating handle and the suction channel is provided with a valve 130, which upon depressing of the suction button admits vacuum from the source 126 into the suction channel and then to the body channel.

The insufflation channel 76 is provided with an inlet port, which is in flow communication with a pressure source 132 provided within a system control unit (SCU) 134. Among the other components of the system control unit 134 a logic unit 136 is shown.

The insufflation channel is provided with an insufflation button 138, which has an opening closeable by the operator' finger. This opening communicates with the insufflation channel such that when the opening is closed pressure can be admitted from the pressure source 132 into the insufflation channel and then to the body channel.

According to the present invention the operating handle is provided with a check valve 140, located at the lateral port so as to be in fluid communication with the suction channel 116 through working channel 124. Thus the check valve 140 senses pressure in the body channel. The check valve is preset to a certain threshold value and thus it will automatically release the inner pressure from the body channel when this pressure exceeds the threshold.

In FIG. 4b is presented still further embodiment of the invention, which refers to the conventional endoscopic apparatus. In this embodiment the same reference numbers as in FIG. 4a designates similar elements and therefore will not be explained again. In this embodiment the operating handle is provided with a means 142 for measuring the pressure and with electrically controlled relief valve 144.

The means 142 for measuring pressure and the relief valve 144 can be fitted with appropriate biologic filter 146,148 for preventing contamination from the channel suction channel 116.

The means 142 senses and measures the inner pressure in the body channel through working channel 124 and suction channel 116. The inner pressure is built during the endoscopic procedure when the body channel is insufflated. For measuring the inner pressure one can use any suitable means, e.g. a pressure transducer or a manometer, capable to provide an output reading corresponding to the inner pressure in the body channel. A solenoid valve can be used as a suitable electrically controllable relief valve.

A first control line 150 is provided, which electrically connects the means 142 for measuring pressure to the logic unit 136. A second control line 152 is provided, which electrically connects the relief valve to the logic unit.

The output reading from the means 142 proceeds to the logic unit where it is permanently monitored and compared with a stored threshold value. If the instant value exceeds the threshold value the logic unit generates a control signal for opening the relief valve.

It should be appreciated that the invention is not limited to the above-described embodiments and that one ordinarily skilled in the art can make modifications or changes without deviating from the scope of the invention, as will be defined in the appended claims. For example, the present invention can be employed not only in the colonoscopic apparatus. It can be used in gastroscopic apparatus or in any other endoscopic apparatus, in which pressure might be supplied to the body channel during the endoscopic procedure and there is a danger that this pressure might cause barotrauma.

It should also be appreciated that the features disclosed in the foregoing description, and/or in the following claims, and/or in the accompanying drawings may, both separately and in any combination thereof, be material for realizing the present invention in diverse forms thereof.

We claim:

1. An endoscopic apparatus for endoscopic examination of a body channel, or a cavity, comprising at least one channel in flow communication with the body channel or cavity, said endoscopic apparatus further comprising a pressure relief arrangement fitted with a valve in flow communication with said channel, said valve preventing an inner pressure within the body channel from exceeding a threshold value.

2. The endoscopic apparatus as defined in claim 1, further comprising an insertion tube provided with at least one channel, an operating handle fitted with a lateral port and a system control unit fitted with a source of pressure, with a source of vacuum, with a logic unit and with appropriate hydraulic and pneumatic components as required for supplying a fluid to the at least one channel.

3. The endoscopic apparatus as defined in claim 2, wherein said the at least one channel comprises a suction channel and said valve being a check valve.

4. The endoscopic apparatus as defined in claim 2, wherein said the at least one channel comprises a suction channel and said valve being an electrically controllable relief valve electrically connected to the logic unit.

5. The endoscopic apparatus as defined in claim 4, wherein said pressure relief arrangement comprises a pressure measuring means electrically connected to the logic unit.

6. The endoscopic apparatus as defined in claim 5, wherein the electrically controllable relief valve being provided with a biological filter.

7. The endoscopic apparatus as defined in claim 6, further comprising a monitor for displaying pressure measured by the pressure measuring means.

8. The endoscopic apparatus as defined in claim 2, wherein said the at least one channel being an insufflation channel and said valve being an electrically controllable relief valve electrically connected to the logic unit.

9. The endoscopic apparatus as defined in claim 8, wherein said pressure relief arrangement comprises a pressure measuring means electrically connected to the logic unit.

10. The endoscopic apparatus as defined in claim 2, wherein said pressure relief arrangement being located at the lateral port of the operating handle.

11. The endoscopic apparatus as defined in claim 10, wherein said pressure relief arrangement comprises a check valve in flow communication with a working channel extending through the lateral port.

12. The endoscopic apparatus as defined in claim 10, wherein said pressure relief arrangement comprises an electrically controllable relief valve electrically connected to the logic unit.

13. The endoscopic apparatus as defined in claim 12, wherein said pressure relief arrangement comprises a pressure measurement means electrically connected to the logic unit.

14. The endoscopic apparatus as defined in claim 13, wherein the relief valve being provided with a biological filter for preventing contamination.

15. The endoscopic apparatus as defined in claim 1, wherein the valve being presettable.

16. The endoscopic apparatus as defined in claim 1, wherein said endoscopic apparatus being a colonoscopic apparatus.

17. The endoscopic apparatus as defined in claim 1, wherein said endoscopic apparatus being a gastroscopic apparatus.

18. The endoscopic apparatus as defined in claim 2, further comprising an inflatable propelling sleeve.

19. A pressure relief arrangement for an endoscopic apparatus used for endoscopic examination of a body channel, said pressure relief arrangement comprising a valve in flow communication with the body channel for preventing an inner pressure within the body channel from exceeding a threshold value.

20. A pressure relief arrangement according to claim 19, further comprising a means for measuring pressure in the body channel during the endoscopic examination.
